(19) United States
(12) Patent Application Publication
(10) Pub. No.: US 2023/0414295 A1
(43) Pub. Date: Dec. 28, 2023

(54) INSTALLATION TOOL FOR INSTALLING A TRACKING MEMBER ON A BONE

(71) Applicants: Stefan W. Kreuzer, Fremont, CA (US); Think Surgical, Inc., Fremont, CA (US)

(72) Inventors: Timothy Pack, Fremont, CA (US); Moran Gera, Fremont, CA (US); Stefan W. Kreuzer, Fremont, CA (US)

(73) Assignee: Think Surgical, Inc., Fremont, CA (US)

(21) Appl. No.: 18/213,398

(22) Filed: Jun. 23, 2023

Related U.S. Application Data
(60) Provisional application No. 63/354,758, filed on Jun. 23, 2022.

(51) Int. Cl. A61B 34/20; A61B 34/10; A61B 34/30

(52) U.S. Cl. CPC A61B 34/20; A61B 34/10; A61B 34/30; A61B 2034/2046; A61B 2034/101

(57) ABSTRACT

An installation tool for installing a tracking member on a substrate is provided that includes a body with at least one guide hole located at a first end of the body for guiding a fixation element into the substrate. A holding mechanism is provided for holding the body to the fixation element. A coupler located at an opposing end of the body is provided for coupling the tracking member to the body. A removable handle has an opening for receiving at least portion of the coupler inside the removable handle. A system is also provide that includes the installation tool and a tracking member.

### RELATED APPLICATIONS

This application is a non-provisional application that claims priority of U.S. Provisional Application Ser. No. 63/354,758 filed Jun. 23, 2022; the contents of which are hereby incorporated by reference.

### FIELD OF THE INVENTION

The invention in general relates to surgery and in particular to tools for installing a tracking member on a bone.

### BACKGROUND

Tracking systems are commonly used in computer-assisted surgical procedures to track various objects in the operating room (OR). These tracked objects may include surgical devices, instruments, and the patient's anatomy. Examples of tracking systems include optical tracking systems and electromagnetic tracking systems. Optical tracking systems generally include a tracking computer that receives data from two or more optical sensors (e.g., cameras) to detect the POSE of three or more fiducial markers (e.g., active markers (e.g., LEDs), passive markers (e.g., retroreflective spheres)) coupled to the tracked object. Three or more fiducial markers arranged in a known geometry may be referred to herein as a tracking array. The optical tracking system is capable of defining a tracking array coordinate system and track movement of the object in six degrees-of-freedom (6-DoF). Electromagnetics tracking systems generally include an electromagnetic field generator for detecting the POSE of electromagnetic sensors coupled to the tracked object, which also enables 6-DoF tracking.

A particular computer-assisted surgical procedure that may utilize an optical tracking system is total knee arthroplasty (TKA). In TKA, the worn articular surfaces of the femur and tibia are removed and replaced with implants to restore the function of the knee so as to alleviate discomfort associated with the worn joint. A TKA begins with the generation of a surgical plan defining one or more locations for a surgical device to perform an action (e.g., removing bone, inserting pins) on the femur and tibia. A femoral tracking array is installed on the femur and the surgical plan is registered to the femur with respect to the coordinate system of the femoral tracking array. A tibial tracking array is installed on the tibia and the surgical plan is registered to the tibia with respect to the coordinate system of the tibial tracking array. This allows the optical tracking system to track movement of the femur and tibia and their respective surgical plans to accurately execute the surgical plan with the surgical device.

Installing a tracking array on a bone generally includes the following. A user identifies a location to install the tracking array on the bone that will not interfere with the removal of the bone or the operation of a surgical device or any other instruments during the procedure. Two fixation elements (e.g., pins, screws) are inserted into the bone at the identified location with the aid of an alignment guide, and a bracket is then fixed onto the fixation elements. The alignment guide includes two guide holes for guiding the fixation elements into the bone, where the guides holes are spaced a distance apart corresponding to a distance between two receiving elements (e.g., holes) of the bracket. After the fixation elements are inserted in the bone, the alignment guide is removed from the bone and discarded. The tracking array is then fixed onto the bracket.

Several problems are associated with this process. First, multiple tools (i.e., alignment guide and bracket) are required to install the tracking array in the bone. This increases the cost of the procedure and increases the overall surgical time. Second, the final location of the tracking array when fixed to the bracket may be sub-optimal (e.g., sub-optimal line-of-sight to the optical detectors, a location which may interfere with the operation of a surgical device). This may require the user to re-insert the fixation elements at a new location on the bone, which requires additional time and additional holes drilled in the bone. Additionally, conventional brackets may have insufficient fixation mechanisms that can cause the bracket to move relative to the fixation elements, or the tracking array to move relative to the bracket. Any movement of the tracking array relative to the bone after registration causes a corresponding movement of the surgical plan relative to the bone, in which event the surgical plan needs to be re-registered to the bone/tracking array before the procedure can resume.

Thus, there exists a need for an installation tool for a tracking member that overcomes the aforementioned limitations.

### SUMMARY OF THE INVENTION

An installation tool for installing a tracking member on a substrate is provided that includes a body with at least one guide hole located at a first end of the body for guiding a fixation element into the substrate. A holding mechanism is provided for holding the body to the fixation element. A coupler located at an opposing end of the body is provided for coupling the tracking member to the body. A removable handle has an opening for receiving at least portion of the coupler inside the removable handle.

A system is also provided that includes the installation tool and a tracking member.

### BRIEF DESCRIPTION OF THE DRAWINGS

The present invention is further detailed with respect to the following drawings that are intended to show certain aspects of the present of invention, but should not be construed as limit on the practice of the invention, wherein:

FIGS. 1A-1C depict an installation tool for a tracking array, where FIG. 1A is a perspective view thereof, FIG. 1B is a front view thereof, and FIG. 1C is a top view thereof;

FIG. 2 is an exploded view of FIG. 1A;

FIG. 3A is a perspective view of the body of the installation tool with the removable handle removed;

FIG. 3B is front view of the body as shown in FIG. 3A;

FIG. 4 is a perspective view of a movable jaw of the holding mechanism;

FIG. 5 is side view of a coupler portion of the body;

FIG. 6 is a perspective view of a removable handle of the installation tool adapted to couple with the coupler;

FIG. 7 is a perspective view of a tracking array; and

FIGS. 8A-8D depict a sequence steps in the usage of the installation tool, where in FIG. 8A, the installation tool is moved to a desired location for inserting fixation elements into the bone, in FIG. 8B, fixation elements are placed through the guide holes and inserted in the bone, in FIG. 8C the removable handle is removed revealing the coupler, and in FIG. 8D, the tracking array is assembled to the coupler.

FIG. 9 depicts an outline of a tracking array positioned relative to a removable handle of the installation tool to show how the location of a user's hand on the removable handle during the installation process will approximate the final location of the tracking array when coupled to the body of the installation tool.

### DETAILED DESCRIPTION OF THE INVENTION

The present invention has utility as a tool and system to efficiently install a tracking array in a substrate. This is especially beneficial during a computer-assisted surgical procedure that utilizes a tracking system. The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Patent documents and publications mentioned in the specification are indicative of the levels of those skilled in the art to which the invention pertains. These documents and publications are incorporated herein by reference to the same extent as if each individual document or publication was specifically and individually incorporated herein by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the terms “computer-assisted surgical device” and “CAS device” refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in U.S. Pat. Nos. 6,757,582; 7,206,626; 8,876,830; and 8,961,536; U.S. Patent Publication No. 2013/0060278; and PCT Patent Publication Nos.: PCT/US2021/031703; and PCT/US2020/062686; which patents and patent application are incorporated herein by reference. The surgical robot may be active (e.g., automatic/autonomous control), semi-active (e.g. a combination of automatic and manual control), haptic (e.g., tactile, force, and/or auditory feedback), and/or provide power control (e.g., turning a robot or a part thereof on and off). The terms “computer-assisted surgical system” and “CAS system” refer to systems utilizing a computer or software to assist in planning a position for one or more bone cuts to be formed on a bone for mounting contact surfaces of an implant thereon, and/or systems that assist in forming one or more bone cuts on a bone. An example of a CAS system may include: i) a CAS device and software used by the CAS device (e.g., cutting instructions); ii) one or more CAS devices (e.g., a surgical robot); and iii) any of the aforementioned with additional devices or software (e.g., a tracking system, tracked/navigated instruments, tracking arrays, bone pins, a rongeur, an oscillating saw, a rotary drill, manual cutting guides, manual cutting blocks, manual cutting jigs, etc.).

Embodiments of the present invention describe a tool and system to efficiently install a tracking member in a substrate. Surgical substrates that commonly require installation of a tracking array include bones and implants. While the present invention is further detailed with respect to a TKA procedure in the accompanying drawings, it is to be understood that the present invention is applicable to computer-assisted surgical procedures in general and regardless of anatomy, as well as manufacturing processes. Examples of such surgical procedures include total and partial joint replacement (e.g., hip, shoulder, ankle, and knee joints); unicompartmental arthroplasty; bone fracture repair; osteotomies; craniotomies; spinal reconstruction; and pedicle screw placement. Exemplary manufacturing processes that benefit from the present invention include composite material part adhesive bead line application and cutting of composite materials. Composite materials are routinely used in the aerospace, vehicle, and sporting goods manufacturing sectors.

The present invention also affords an additional advantage in allowing a surgeon or a biomechanical engineer to simulate operation of a replaced joint through the relative movement of tracking arrays. As a result, refinements of the TKA can occur through ligament adjustment or soft tissue adjustments.

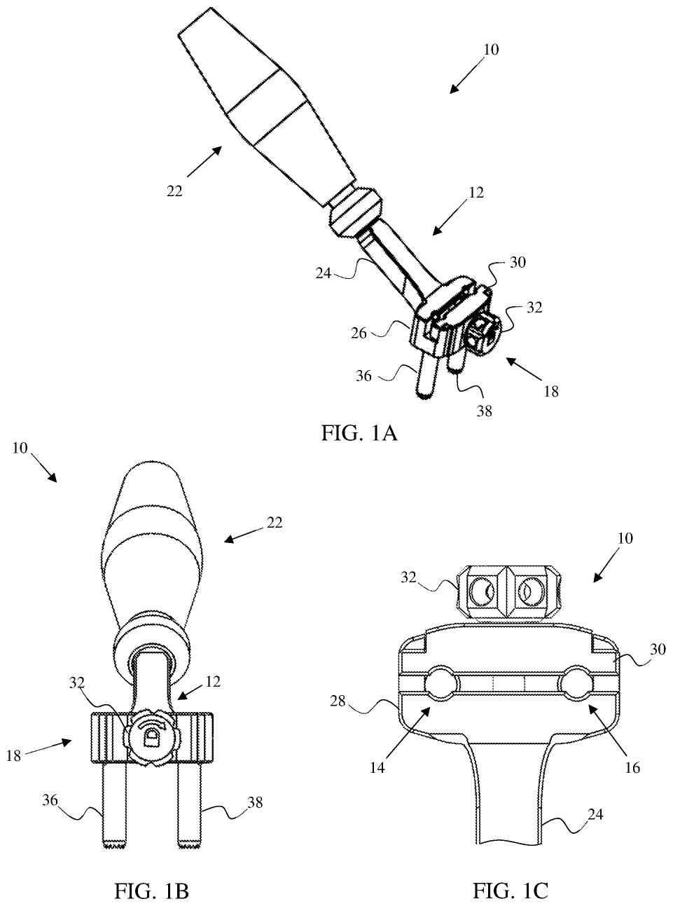

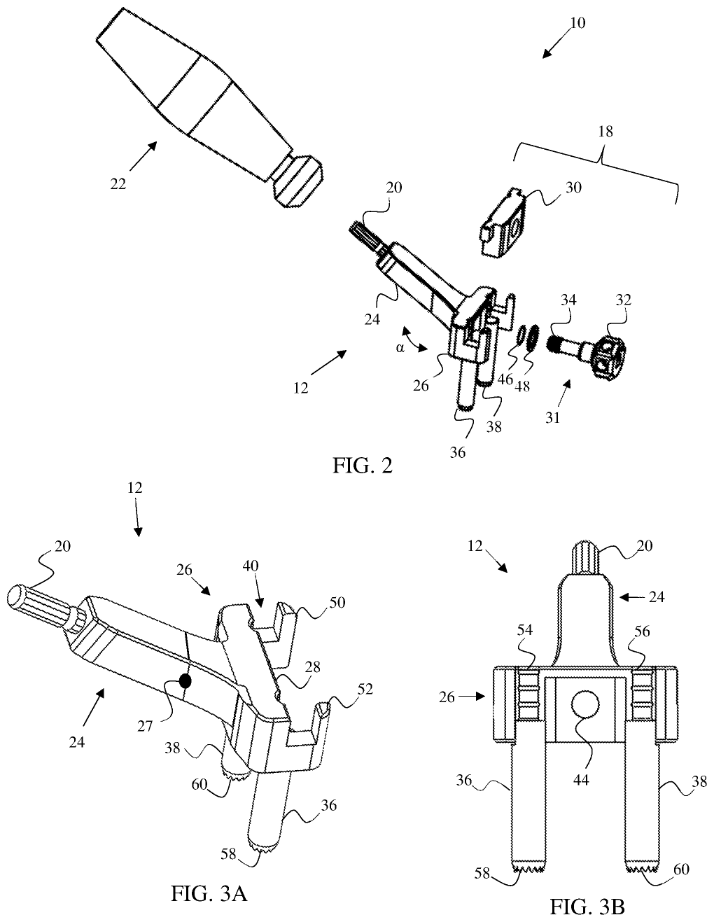

Referring now to the drawings, FIGS. 1A-1C depict an installation tool 10 for a tracking array, where FIG. 1A is a perspective view thereof, FIG. 1B is a front view thereof, and FIG. 1C is a top view thereof. The installation tool 10 generally includes a body 12, at least one guide hole (14, 16), a holding mechanism 18, a coupler 20 (as best shown in FIGS. 2, 3A, 3B, and 5), and a removable handle 22. While the guide holes (14,16) are depicted as circular in cross-section and of similar length, it is appreciated that in various embodiments, these each vary independently in dimensions and shape. The guide hole(s) (14, 16) may be located at a first end of the body, where each guide hole (14, 16) is independently configured to guide a fixation element (e.g., pin, screw) into the bone. It is further appreciated that non-degenerate fixation elements adapted to affix a tracking array assures a single mounting orientation thereof. The holding mechanism 18 may be located at a first end of the body 12 and is configured to hold the body 12 to the fixation elements inserted in the bone. The coupler 20 (as shown in FIG. 2) may be located at an opposing end of the body 12 and is configured to couple a tracking member (e.g., a tracking array, an electromagnetic sensor) to the body 12. The removable handle 22 includes an opening 23 (as shown in FIG. 6) for receiving at least a portion of the coupler 20 inside the removable handle 22.

The installation tool 10 provides several advantages relative to conventional tools, these advantages include the fact that the installation tool 10 is an all-in-one tool for installing a tracking member to the bone. The installation tool 10 guides the fixation elements into the bone or other substrate and directly couples to the fixation elements, where a tracking member can be coupled to the body 12 of the installation tool 12 via the coupler 20 upon removal of the removable handle 22. Further, the removable handle 22 provides the user with feedback as to the final location of the tracking member because the location of the user's hand on the removable handle 22 during the installation process can approximate the final location of the tracking array when coupled to the coupler 20. This feedback is depicted in FIG. 9, which shows how the location of a user's hand on the removable handle 22 during the installation process will approximate the final location of the tracking array (shown as outline 100′ relative to the removable handle 22) when coupled to the body 12 of the installation tool 10.

The body 12 may include a first portion 24 and a second portion 26. The first portion 24 may be angled relative to the second portion 26 to position the tracking member at an optimal view to the tracking system. The first portion 24 may be elongated. The second portion 26 may be part of the holding mechanism 18 and located at the first end of the body 12. The second portion 26 may be substantially planar relative to the first portion 24, with the first portion 24 angled “a” anywhere from 1 to 90 degrees from the second portion 26, but preferably in the range of 20 to 70 degrees. In some inventive embodiments, a lockable hinge 27 is provided to allow the first portion to pivot relative to the second portion 26 to adjust the angle “a”. As a result, a tracking array mounted thereto can be repositioned for reasons that illustratively include alignment with other tracking system components or better access to the surgical field.

The holding mechanism 18 may include a stationary jaw 28 and a movable jaw 30 to clamp the body 12 to the fixation elements inserted in the bone. The movable jaw 30 may be moved relative to the stationary jaw 28 with a tightening element (e.g., by rotating a knob 32 coupled to a screw 34 (as shown in FIG. 2)). It should be appreciated that other holding mechanisms 18 may be used including other clamping configurations, clasps, set-screws, and the like.

The guide hole(s) (14, 16) are positioned between the stationary jaw 29 and the movable jaw 30. The guide hole(s) (14, 16) may be bored through a portion of the body 12 such as the second portion 16 of the body 12. Guide tubes (36, 38) may extend beyond the guide holes (14, 16) for further guidance and stabilization of the fixation elements during installation. In other embodiments, the guide tubes (36, 38) having guide holes (14, 16) are coupled to the body 12 (rather than bored through a portion of the body 12), and more particularly coupled to the second portion 26 of the body 12 between the stationary jaw 28 and the movable jaw 30. It should be appreciated that the term “couple” or “coupled” as used herein refers to a connection, an attachment, a link, a join, or an integration of one component to another, either directly or indirectly.

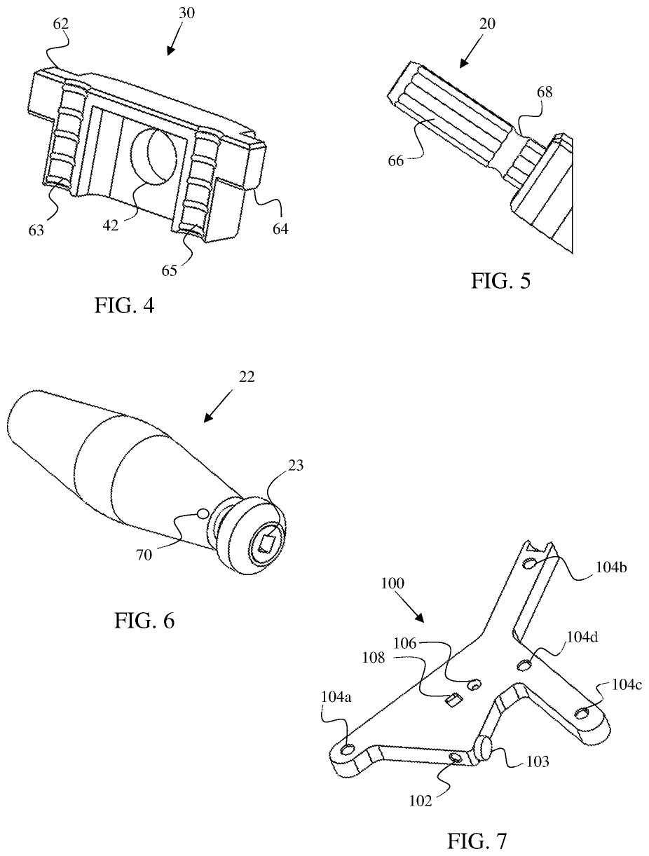

FIG. 2 is an exploded perspective view of the installation tool 10. The removable handle 22 has an opening 23 (as shown in FIG. 6) to receive the coupler 20 inside the opening 23 such that at least a portion of the coupler 22 resides inside the removable handle 22 when the removable handle 22 is assembled to the installation tool 10. The movable jaw 30 may assemble to the second portion 26 of the body 12 in a slot 40 (as shown in FIG. 3A) formed in the second portion 26. A tightening element 31 comprising a knob 32 coupled to a screw 34 may fit through a hole 42 in the movable jaw 30 (as shown in FIG. 4) and a hole 44 in the body 12 (as shown in FIG. 3B) to assemble the movable jaw 30 to the body 12 and to cause the movable jaw 30 to move relative to the stationary jaw 28 upon tightening and loosening the tightening element 31. The holding mechanism 18 may further include other hardware (46, 48) (e.g., washers, nuts, springs, etc.) associated with the tightening element 31.

FIGS. 3A and 3B depict the body 12 of the installation tool 10, where FIG. 3A is a perspective view thereof, and FIG. 3B is a front view thereof. The body 12 may include the first portion 24 and the second portion 26. A coupler 20 may be coupled to a first end of the first portion 24, while the opposing end of the first portion 24 is coupled to the second portion 26. The coupler may be a projection projecting from the first end of the first portion 24. The coupler 20 may be elongated as shown or be a threaded hole in the first portion 24. As shown in FIG. 7, an opening 102 in a tracking array 100 (or any other tracking system member) couples to the coupler 20 to attach the tracking array 100 to the body 12. The second portion 26 includes a slot 40 to receive the removable jaw 30. The slot 40 may be formed with one or more capturing members (50, 52) (e.g., ‘L’ shaped member, hooks) extending from the stationary jaw 28. The stationary jaw 28 may further include in some inventive embodiments, one or more channels (54, 56) (as shown in FIG. 3B) for mating with an outer surface of a fixation element. The one or more channels (54, 56) may have a shape complementary to the shape of a portion of the outer surface of the fixation element. For example, the channels (54, 56) may be semi-circular, or arced, to conform to a surface of a cylindrical fixation element. The guide tubes (36, 38) may further include teeth (58, 60), or a jagged edge, at the end of the guide tubes (36, 38) for gripping the bone during the installation process. The second portion 26 may further include the hole 44 for receiving a portion of the tightening element 31. The hole 44 may be threaded to cause a screw 34 of the tightening element 31 to tighten or loosen upon rotating the tightening element 31 via the knob 32.

FIG. 4 depicts the movable jaw 30. The movable jaw 30 may include one or more tabs (62, 64) for catching the capturing members (50, 52) associated with the slot 41 of the second portion 26 of the body 12. The movable jaw 30 may further include one or more channels (63, 65) complementary to the one or more channels (54, 56) of the second portion 26 of the body for mating with an outer surface of a fixation element. The movable jaw 30 further includes a hole 42 for receiving the tightening element 31 to assemble the movable jaw 30 to the body 12. The hole 42 may or may not be threaded to interact with a screw of the tightening element 31.

FIG. 5 depicts the coupler 20 in a magnified side view. The coupler 20 may be a projection projecting from a first end of the first portion 24 of the body 12. The coupler 20 may have a plurality of facets. In particular embodiments, the coupler 20 has at least 8 facets, while in other embodiments the coupler 20 has 10+ facets. In a particular embodiment, the coupler 20 has 12 faces (i.e., a cross-section of the coupler 20 is a dodecagon). A tracking member may use a set-screw to secure the tracking member to a facet of the coupler 20. Having more facets on the coupler provides the user with greater flexibility or options to orient/rotate the tracking member to a desired orientation on the coupler 20, where the set-screw will make contact with a flat facet of the coupler 20. In other words, there are 12 different orientations that the tracking member can be oriented on the coupler 20 where the set-screw will make contact with a flat facet of the coupler 20. The coupler 20 may further include a groove 68. The groove 68 may couple with one or more components of coupling mechanism 70 associated with the removable handle 22. For example, the groove 68 may receive a portion of a ball, where the ball is a component of a ball-detent mechanism associated with the removable handle 22. The removable handle 22 may therefore be held onto the coupler 20 until it needs to be removed.

FIG. 6 depicts the removable handle 22. The removable handle 22 includes the opening 23 for receiving and/or selectively attaching to at least a portion of the coupler 20 inside the removable handle 22. The opening 23 may be a slot or a channel for receiving the coupler 20 therein. The removable handle 22 may further include a coupling mechanism 70 (e.g., a ball-detent mechanism, a latch, a clamp, a set-screw, etc.) for holding and releasing the removable handle 22 to/from the coupler 20.

FIG. 7 depicts a tracking array 100. The tracking array 100 may include a plurality of fiducial markers 104, an optional data transmitting light-emitting-diode (LED) 106, an optional photosensor 108, an opening 102, and tightening mechanism 103. The fiducial markers 104 may be active markers (e.g., LEDs) or passive markers (e.g., retroreflective spheres). The data transmitting LED and photosensor may transmit and receive data as described in U.S. Pat. No. 11,229,487; assigned to the assignee of the present application and incorporated by reference herein. The opening 102 may receive at least a portion of the coupler 20 in the tracking array 100. The tightening mechanism 103 may include a set-screw coupled to a knob for coupling the tracking array 100 to the coupler 20. For example, a user may slide the tracking array 100 onto the coupler via the opening 103 and then rotate the knob/set-screw onto a face of the coupler 20, thereby coupling the tracking array 100 to the coupler 20. It should be appreciated that the coupling between the holding mechanism 18 and the fixation elements, and the coupling between the coupler 20 and the tracking member 100, forms a rigid and fixed connection. Other examples for securing the tracking array 100 to the coupler 20 include fasteners, latches, clips, clasps, a press-fit of the opening 102 with the coupler 20, an interaction fit of the opening 102 with the coupler first threads in the opening 102 to interact with second threads on the coupler 20.

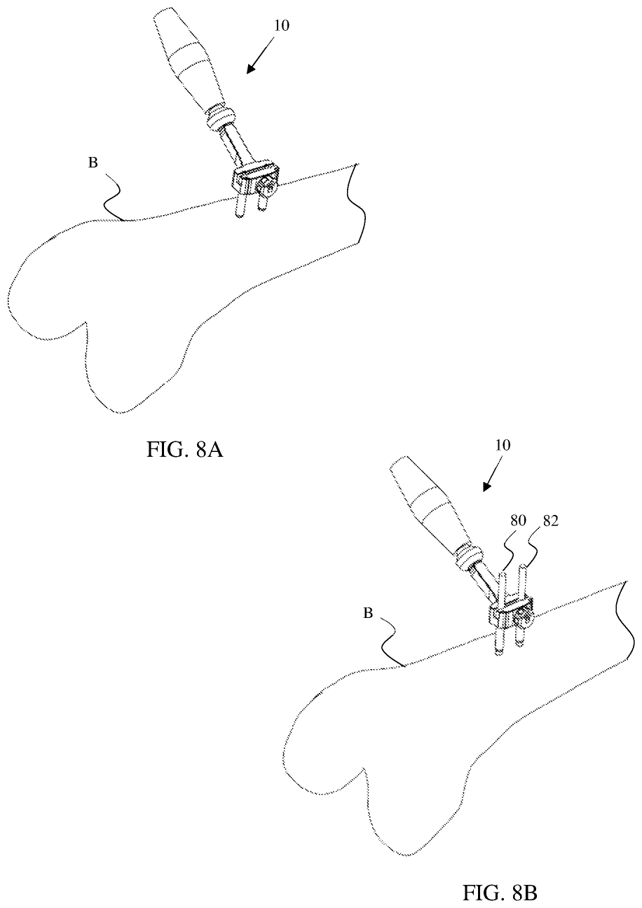

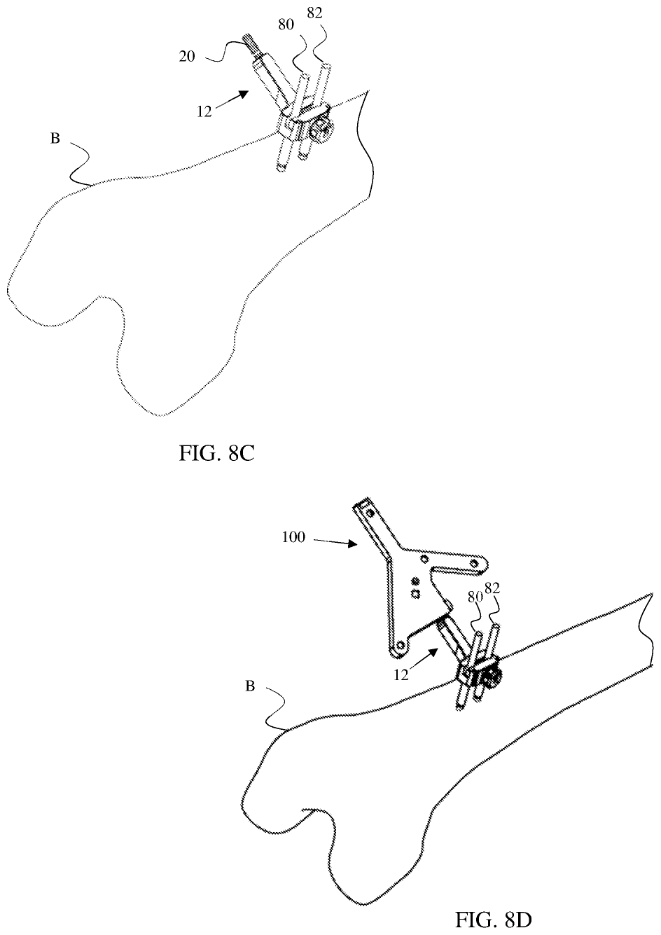

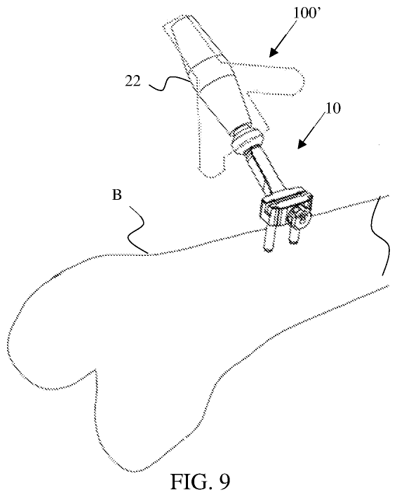

FIGS. 8A-8D depict a method for installing a tracking array (or other tracking member) onto a bone with the installation tool 10. In FIG. 8A, the installation tool 10 is moved to a desired location for inserting fixation elements into the bone ‘B’. While the position of the guide holes (14, 16) will indicate the position for the fixation elements, the position of the removable handle 22 will also indicate the final position for the tracking array. In FIG. 8B, fixation elements (80, 82) are placed through the guide holes (14, 16) and inserted in the bone B′. The holding mechanism 18 is then secured to the fixation elements (80, 82) to hold the body 12 to the fixation elements (80, 82). In FIG. 8C, the removable handle 22 is removed from the installation tool 10 revealing the coupler 20. In FIG. 8D, a tracking array 100 is assembled to the coupler 20 by sliding the tracking array 100 onto the coupler 20 via the opening 102 of the tracking array 100 and then securing the tracking array 100 to the coupler 20 via the tightening mechanism 103.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. An installation tool for installing a tracking member on a substrate, comprising:
a body;
at least one guide hole located at a first end of the body for guiding a fixation element into the substrate;
a holding mechanism for holding the body to the fixation element;
a coupler located at an opposing end of the body for coupling the tracking member to the body; and
a removable handle having an opening for receiving at least portion of the coupler inside the removable handle.

2. The tool of claim 1 wherein the substrate is bone and further comprising the fixation elements.

3. The tool of claim 1 wherein the tracking member is adapted to be attached to the coupler upon removal of the removable handle.

4. The tool of claim 1 wherein the body comprises a first portion defining an angle relative to a second portion wherein the second portion is part of the holding mechanism and located at first end of the body.

5. The tool of claim 4 wherein the angle is from 0 to 90 degrees.

6. The tool of claim 1 wherein the holding mechanism comprises a stationary jaw and a movable jaw to clamp the body to the fixation element inserted in the substrate.

7. The tool of claim 6 wherein the movable jaw is moveable relative to the stationary jaw with a tightening element.

8. The tool of claim 6 further comprising at least one guide hole positioned between the stationary jaw and the movable jaw.

9. The tool of claim 8 wherein one of the at least one guide hole is elongated to form a guide tube.

10. The tool of claim 9 further comprising teeth or a jagged edge at an end of the guide tube for gripping the substrate.

11. The tool of claim 1 wherein an opening in the tracking member is adapted to couple to the coupler when the removeable handle is displaced from the coupler.

12. The tool of claim 6 wherein the stationary jaw further comprises one or more channels adapted for mating with an outer surface of the fixation element.

13. The tool of claim 6 wherein the movable jaw further comprises one or more tabs.

14. The tool of claim 1 wherein the coupler further comprises a plurality of facets.

15. The tool of claim 1 further comprising a set screw adapted to simultaneously engage the coupler and the tracking member, a groove adapted to engage the removable handle, or a combination thereof.

16. The tool of claim 1 wherein the removable handle further comprises a coupling mechanism for holding and releasing the removable handle to/from the coupler.

17. A system comprising:
the installation tool of claim 1; and
a tracking member.

18. The system of claim 17 wherein the removable handle, during the installation process, approximates the final location of the tracking member when coupled to the coupler.

19. The system of claim 17 wherein the tracking member further comprises at least one of a fiducial marker, a data transmitting light-emitting-diode, a photosensor, or a tightening mechanism.

20. The system of claim 17 wherein the tracking member is adapted to be attached to the coupler upon removal of the removable handle.
